(19) United States
(12) Patent Application Publication
(10) Pub. No.: US 2019/0223978 A1
(43) Pub. Date: Jul. 25, 2019

(54) COMPOSITIONS AND METHODS FOR INHIBITING CONTAMINATION IN SPLASH BASINS

(71) Applicant: Innovation Technologies, Inc., Lawrenceville, GA (US)

(72) Inventor: Peter GRAVES, Lawrenceville, GA (US)

(21) Appl. No.: 16/255,299

(22) Filed: Jan. 23, 2019

Related U.S. Application Data
(60) Provisional application No. 62/620,722, filed on Jan. 23, 2018.

(51) Int. Cl. A61B 90/70; A61L 2/18; B65D 23/06

(52) U.S. Cl. CPC A61B 90/70; A61L 2202/24; B65D 23/06; A61L 2/18

(57) ABSTRACT

The problem of contamination of sterile splash basins and surgical instrumentation used in operating rooms is solved by the use of an aqueous composition containing chlorhexidine gluconate in a concentration of sufficient strength to provide inhibition of bacterial growth and disinfection of surgical instruments, but is not harmful to non-mucosal tissues. Pre-filled bottles of the aqueous composition are also provided, with drip-free mouths that minimize contact with the exterior of the bottle. Also disclosed are pre-filled splash basins with removable covers that help maintain sterility. Also disclosed are pre-filled ampoules in sterile wrappers for dilution into sterile splash basins with dispensed sterile water.

### CROSS REFERENCE TO A RELATED APPLICATION

This application claims the benefit of U.S. Provisional Application Ser. No. 62/620,722, filed Jan. 23, 2018, which is incorporated herein by reference in its entirety.

### BACKGROUND OF INVENTION

In an operating room, instruments are usually soaked, wiped, or rinsed in sterile water during a surgical procedure to inhibit blood, tissue, and other contaminants from drying and caking, which can make later cleaning and sterilizing more difficult.

Soaking is typically performed in a sterile “splash basin” kept in the operating room, typically in a single or double ring stand. As instruments are used, they are often placed in the splash basin to be covered with the sterile water. If necessary, instruments are removed from the splash basin for reuse and then returned to the splash basin. When instruments are reused, they are taken directly from the splash basin to the patient.

Studies have shown that splash basins in operating rooms can be a significant source of contamination. (Baird, Robert A., M.D., et al., “Splash Basin Contamination in Orthopaedic Surgery” Clinical Orthopaedics and Related Research (Section II: General Orthopaedics), July/August, 1984.) Splash basins are often kept near the surgical back table, but far away from the operating field. This can lead to increased potential for contamination. It has also been shown that contaminated splash basins result in cross-contamination of instruments and, if reused, can introduce contamination into the operating field and patient. (Anto, Benny, et al. “Splash Basin bacterial contamination during elective arthroplasty” March 2006, J. of Infection, Vol. 52, issue 3, pgs. 231-232)

While there are numerous products available that can be applied to soiled instruments that assist in breaking down the blood and other materials on the instruments, they are typically not used in sterile splash basins because they are not considered safe for contact with delicate internal tissues. They are typically applied to the soiled instruments after the surgical procedure is complete. Thus, using them to decant sterile solutions into a splash basin can introduce contaminants into the splash basin as well as the operating theater where they would be decanted. This leaves few, if any, effective options for controlling or eliminating contamination in splash basins.

### BRIEF SUMMARY

The subject invention provides devices and methods for controlling or eliminating contamination of splash basin fluids used in operating rooms. More specifically, embodiments of the subject invention provide compositions that are effective in controlling contamination of splash basin fluids and instruments therein, as well as containers for decanting the compositions or splash basins that can sterilize compositions decanted therein. In some embodiments, the structure of the containers and features thereof inhibit introduction of contaminants from the outside of the container into a splash basin. In certain other embodiments, the containers have drip-less or drip-free pour spouts or lips that prevent drips from the non-sterile outside of the container from reaching the splash basin. Alternative embodiments include splash basins that are pre-filled with compositions or that are coated with compositions that dissolve in water for sterilizing the splash basin fluids. In other alternatives, a concentrated CHG solution is provided in a container that can be opened and decanted into sterile water bottles to create a diluted composition for use in a splash basin or directly into a splash basin filled with sterile water to create a diluted composition.

In one embodiment, a low concentration solution of chlorhexidine gluconate (CHG) can be used in one or more of the splash basins often utilized in an operating room. The concentration of CHG is sufficient to be an antimicrobial and a disinfectant, but not high enough to harm internal tissues. The use of CHG in a splash basin safely inhibits or eliminates contamination, thereby making it possible to reuse surgical tools without concern for either infectivity or toxicity to tissues.

Advantageously, CHG is active against gram-positive and gram-negative organisms, facultative anaerobes, aerobes, and some yeasts. CHG can be more advantageous to use than antibiotics because it operates by mechanical control of organisms. This reduces the selection pressures that give rise to resistant organisms. At the advantageous concentrations described herein, CHG can be both bacteriostatic and bactericidal. In accordance with the subject invention, at non-toxic concentrations, CHG causes an alteration in bacterial cell osmotic equilibrium, causing the microbial cells to “leak” cellular contents, thereby killing the microbes within a short period of time.

Many products that contain CHG are used externally and often include ethanol or isopropyl alcohol. CHG products are counter-indicated for use in eyes, ears, or in contact with the meninges. Many prescription and over-the-counter products can also contain various other substances or materials that aid or improve the use of the products on skin or in the mouth, such as aloe, glycerin, castor oil, or propylene glycol. These additives can be harmful to internal tissues and can also coat instruments, making them hard to use, toxic to tissues and/or causing damage to the instrument surfaces, when subjected to more thorough cleaning procedures. In addition, many recommending bodies warn against using these types of products for the above mentioned reasons.

Splash basins are common in most operating rooms. They are used to allow for soaking of instrumentation in order to prevent blood, body fluids, and fats, or other debris from caking and drying on surgical instruments, which can inhibit their reuse. Allowing these materials to dry on surgical instrumentation can make them harder to clean and sterilize later.

While useful, splash basins have been linked to increased infections. Several studies have shown that they can become contaminated with a variety of microorganisms. The subject invention provides sterilizing and disinfecting compositions that can be used in splash basins, thereby reducing contamination. Specific embodiments provide pre-filled containers in which the CHG composition can be stored and dispensed.

Embodiments of the subject invention utilize CHG in sterile water as an antimicrobial and disinfecting composition. In further embodiments, the CHG/sterile water composition is used in reducing contamination of surgical instruments placed within a splash basin. Advantageously, the concentration necessary to achieve sterilization and disinfection of instruments has been demonstrated to be non-harmful to tissues, even internal, non-epithelial tissues, when the instruments are reused directly from the splash basin

Certain other embodiments of the subject invention provide ampules, containers or bottles for dispensing the CHG/sterile water composition. The ampules, containers, or bottles can have structural features that inhibit contamination when dispensing. The ampules, containers, or bottles can have, for example, drip-free openings or lips, or twist-off neck with flanges. Pre-filled or CHG coated disposable splash basins are also disclosed.

### BRIEF DESCRIPTION OF DRAWINGS

In order that a more precise understanding of the above recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed. Any variations of these dimensions that will allow the subject invention to function for its intended purpose are considered to be within the scope of the subject invention.

FIG. 1A shows a non-limiting example of a hospital approved bottle and separate lid in which a composition, according to the subject invention can be stored.

FIG. 1B (prior art) shows an example of a radially flared edge that forms a drip-free curved surface.

FIG. 1C (prior art) shows an example of a cut-out around the exterior of the mouth of a bottle to form a drip-free lip.

FIG. 1D shows a bottle having a sharp edge around the mouth that inhibits drips and minimalizes contact of the decanted liquid with the exterior of the bottle.

FIGS. 2A, 2B, and 2C show non-limiting examples of splash basins pre-filled with a composition, according to the subject invention. FIG. 2A shows a pre-filled splash basin (PFS basin) with a peel-away lid. FIG. 2B shows an example of a splash basin after a lid is removed showing the composition in the basin ready for use. FIG. 2C shows an example of a PFS basin with a sealable lid in place over a used splash basin.

FIGS. 3A, 3B and 3C show non-limiting examples of non-round PFS basins. FIG. 3A shows a non-round splash basin with a peel-away lid that is also enclosed in a wrapping. FIG. 3B shows a non-round splash basin with the lid removed and the composition already therein ready for use. FIG. 3C shows a peel-away lid with an adhering material for reattachment.

FIG. 4 illustrates a typical ring-stand used to support splash basins.

FIGS. 5A and 5B illustrate ampoules for containing a concentrated CHG. FIG. 5A illustrates an example of an ampoule with a twist-off neck. FIG. 5B illustrates an example of a vial with a screw-off lid.

FIG. 6 illustrates an example of a PCS basin with a coating of CHG on the bottom inside.

### DETAILED DISCLOSURE

The subject invention provides devices, as well as compositions and methods that utilize chlorhexidine gluconate (CHG) for disinfecting surgical instruments. More specifically, the subject invention provides compositions and methods for use in splash basins utilized in operating rooms to soak and rinse surgical instruments. Specific embodiments utilize CHG in sterile water. In a more specific embodiment, the composition does not contain an alcohol. Further embodiments include containers for dispensing CHG solutions and splash basins pre-filled with CHG solution or coated with CHG that can be made into a solution by the addition of sterile water or other fluid.

The subject invention is particularly useful in the field of orthopedic surgical procedures, during which surgical instruments can be reused during a procedure. The materials and methods of the subject invention can also be used in surgeries of the gastrointestinal tract, cardiovascular system, or with other areas or systems of a body. This does not preclude embodiments of the subject invention being used in other situations and for other types of surgical procedures where disinfection of surgical instruments can be beneficial.

In the description that follows, a number of terms used in relation to surgical or medical procedures are utilized. In order to provide a clear and consistent understanding of the specification and claims, including the scope to be given such terms, the following definitions are provided:

The term “patient” as used herein, describes an animal, including mammals, to which the devices and methods of the present invention can be applied and that can benefit from such application. This can include mammalian species such as, but not limited to, apes, chimpanzees, orangutans, humans, monkeys; domesticated animals (e.g., pets) such as dogs, cats, guinea pigs, hamsters; veterinary uses for large animals such as cattle, horses, goats, sheep; and, any wild or non-domesticated animal. Human or non-human animal patients can range in age from neonates to elderly.

The terms “container” or “bottle” as used herein is merely for literary convenience. A container or bottle can be any receptacle, bowl, ampoule, or other type of hollow body for holding, storing and decanting a composition and to which the embodiments of the subject invention can be applied.

Also, as used herein, and unless otherwise specifically stated, the terms “operable communication,” “operable connection,” “operably connected,” “cooperatively engaged” and grammatical variations thereof mean that the particular elements are connected in such a way that they cooperate to achieve their intended function or functions. The “connection” or “engagement” may be direct, or indirect, physical or remote.

The present invention is more particularly described in the following examples that are intended to be illustrative only because numerous modifications and variations therein will be apparent to those skilled in the art. As used in the specification and in the claims, the singular for “a,” “an” and “the” include plural referents unless the context clearly dictates otherwise.

Splash basins are commonly used in operating rooms to soak and rinse soiled surgical instruments. These basins are often susceptible to contamination and can contribute to post-operative infections. Instruments are deposited and removed from the splash basin, as necessary, during a procedure. Any contaminated instrument placed in the splash basin can result in cross-contamination of all the instruments in the splash basin.

Splash basins are typically filled with sterile water at the beginning of a surgical procedure. The Association for the Advancement of Medical Instrumentation (AAMI) and the Association of periOperative Registered Nurses (AORN) recommend that sterile water be used in splash basins. Sterile water is preferred as it does not damage instruments, as saline, alcohol, or other substances often can. Instruments removed from the splash basin are often handed directly to the surgeon, with minimal or no wiping before presentation. Any sterile water on the instrument does not cause harm to the tissue, unlike other compositions used for disinfecting or sterilizing instruments. Thus, in an effort to protect instruments and patients, sterile water has been the recommended option for use in splash basins.

Chlorhexidine gluconate (CHG) has broad-spectrum efficacy. It has high antimicrobial activity, including activity against MRSA and other antibiotic resistant organisms.

CHG is active against gram-positive and gram-negative organisms, facultative anaerobes, aerobes, yeasts, and some viruses. Advantageously, CHG can also have activity against biofilms. CHG operates by mechanical control of organisms, which can reduce the selection pressures that give or are known to give rise to resistant organisms. At the advantageous non-toxic concentrations described herein, CHG can be both bacteriostatic and bactericidal and causes an alteration in bacterial cell osmotic equilibrium, thereby killing the organism.

CHG has a high affinity for binding to skin, mucus membranes, oral mucosa, and other epithelial tissues and has a residual effect. As such, compositions that include CHG usually employ it at lower concentrations and combine it with alcohol (ethanol and isopropyl), as it has been assumed that lower CHG concentrations are not as effective. Where CHG is combined with water, it is usually recommended that higher concentrations be employed, often as much as between 5%-10% w/v, to inhibit microbial contamination. At this concentration, aqueous solutions of CHG are typically utilized for sterilization and disinfection of instruments, but are counter-indicated for use on or within the body.

Advantageously, lower concentrations of CHG are effective not only as a disinfectant and sterilizer for instruments, but have been demonstrated to have minimal or no effect on non-epithelial tissues.

In one embodiment, CHG is employed as a pre-mixed fluid at a concentration of approximately 1% w/v or less. In a more particular embodiment, the CHG fluid composition has a concentration of CHG that is less than 0.5% w/v, 0.1% w/v, 0.05% w/v, or 0.03% w/v, or a w/v in a range between any of the listed values.

In a further embodiment, CHG is pre-mixed with water. The water can be obtained through, for example, distillation, deionization, or reverse osmosis procedures. Each of these processes produces a different water product with different constituents therein. Thus, it may be preferable to select one or the other for certain conditions or procedures. In a further embodiment, the water is sterilized.

In another embodiment, CHG can be provided in a concentrated form, such as, but not limited to, liquid, powder, or granules. In one embodiment, the concentrated CHG is provided in pre-measured amounts. The pre-measure amount can be added to another fluid to obtain a diluted CHG composition that has a concentration of CHG that is approximately 1% w/v or less. In a particular embodiment, the concentrated CHG, when mixed and/or diluted with another fluid to provide a composition having a CHG concentration of less than 0.5% w/v, 0.1% w/v, 0.05% w/v, or 0.03% w/v, or a w/v in a range between any of the listed values.

Because the CHG composition can come into contact with patient tissues, additional constituents that can harm tissue, inhibit healing, or have any other deleterious effects are to be avoided. In one embodiment, the composition is CHG and sterile water. In an alternative embodiment, the composition has at least one additional constituent. Preferably, the at least one additional constituent is non-harmful to patient tissues.

In one embodiment, an additional constituent is any of a variety of dyes, known to those with skill in the art, and generally regarded as safe (GRAS). Dyes can provide a helpful indication to a surgeon that an instrument has been removed from the splash basin for reuse. A dye can also be used to highlight or make visible areas on the instrument where debris may still be present because of insufficient or inadequate rinsing.

In another embodiment, an additional constituent is any of a variety of GRAS surfactants. Surfactants can aid in cleaning and rinsing of debris from instruments. Thus, instruments removed from the splash basin having a surfactant can be cleaner and have less debris.

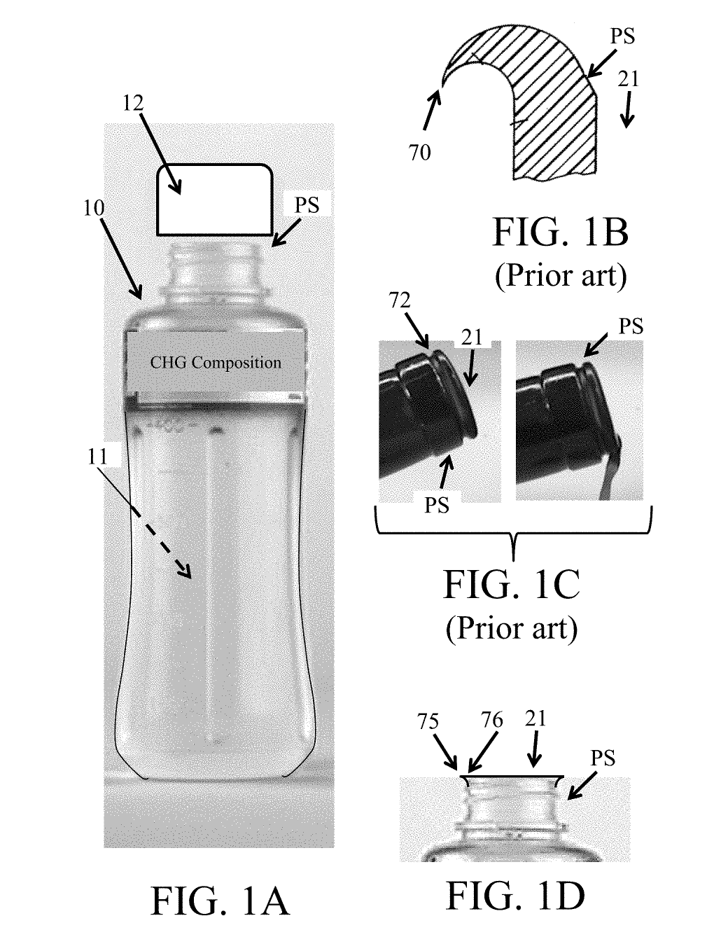

Currently, splash basins are filled with one to two liters of sterile water packaged in bottles or other containers. It is not unusual that several bottles of sterile water are used to fill a splash basin. The CHG compositions of the subject invention can also be stored and decanted from hospital-approved bottles 10 in a similar manner. FIG. 1 illustrates an embodiment of a hospital-approved bottle with an interior 11 and a removable closure 12 in which a CHG composition 15 could be stored. These bottles can be part of a single use, disposable system. In one embodiment, the bottles have a non-gurgle design, which allows the composition to flow smoothly, with minimal or no back-splashing, from the bottle opening. The removable closure can be replaceable on the bottle, such as, by way of example, a screw-top lid, or permanently removable, such as, for example, a snap-off or break-away lid. A person of skill in the art can determine the appropriate type of removable closure.

In an alternative embodiment, the bottles or other containers in which the CHG composition is decanted have a drip-free edge or drip-free lip. The drip-free edge or drip-free lip can inhibit the composition from carrying contaminants that may be on the exterior of the non-sterile bottle or container from being carried to the splash basin. In one embodiment, the drip-free edge or drip-free lip is molded or formed as part of the bottle or container. In another embodiment, the drip-free edge or drip-free lip is part of an attachment that is added to the container before decanting.

Splash basins can have a capacity of several liters, with some holding nine liters or more. Typical sterile water bottles can hold about a liter. Typically one or two liters are utilized during most procedures. Procedures for introducing a container into the sterile field of an operating room can be time-consuming. A pre-filled splash basin can undergo procedures once and have a fully filled splash basin ready for use.

FIG. 1A illustrates a non-limiting example of a type of bottle that can be used to contain and store a chlorhexidine gluconate (CHG) composition, as described above. A bottle of CHG to be used for filling a splash basin can hold any amount of CHG composition. It can be preferable for a bottle to contain an amount of CHG composition that minimizes the number of bottles required to fill a splash basin, but not contain so much that the weight makes it unwieldy or unsafe to hold and manipulate. In one embodiment, a bottle containing a CHG composition of the subject invention can contain between about 250 ml. and about cone liter. In a further embodiment, a bottle containing a CHG composition of the subject invention can contain between about 500 ml and about 750 ml. In a particular embodiment, a bottle containing a CHG composition of the subject invention can contain about 500 ml.

Certain embodiments of the subject invention provide improvements to bottles used for decanting CHG solution. More specifically, embodiments of the subject invention provide bottles with structures or features that inhibit the contents, when being decanted, from coming into contact with the exterior of the bottle. Specific embodiments include openings or mouths that have drip-free lips that inhibit the contents from contacting the potentially-contaminated exterior of a bottle and then dripping into a splash basin.

In one embodiment, a drip free lip is configured as part of the bottle, such that, when the bottle is opened the drip-free lip is ready to use. In an alternative embodiment, a drip-free lip is part or a component of an attachment(s) that can be placed on or attached to the bottle, either before or after opening. Drip-free lips and edges are known in the art and used for decanting a variety of substances from different types of bottles. They typically operate by providing some point beyond which decanting liquids cannot pass or cross to migrate down the outside of the bottle or pour spout. The closer the drip-less lip is to the pour-spout, the less contact the decanting liquid has with the exterior of the bottle.

One example of a drip-free lip that can be utilized with embodiments of the subject invention is disclosed in U.S. Pat. No. 5,215,694. The '694 patent teaches methods and devices for forming a tapered sharp edge on a pour spout PS. The tapered sharp edge is engaged with a tool that both heats and causes the sharp part of the edge to be outwardly radially folded or molded to form a curved surface, as shown in FIG. 1B. The curved surface performs as a drip-free lip on the mouth 21 of the bottle.

Another example of a drip-free lip is shown in FIG. 1C, wherein the edge of the mouth 21 of the pour spout PS on a bottle is formed with a radial cut-out 72 around the pour spout and just beneath the mouth or opening. The radial cut-out forms a depression that inhibits liquid being decanted from the mouth from migrating down the neck of the bottle. Any drips on the edge of the bottle that remain as the bottle is tilted upwards are directed back into the bottle.

Yet another example of a drip-free lip is shown in U.S. Pat. No. 5,597,090 which utilizes a channel formed in the pour spout for capturing any final liquid that may be present on the pour spout when the container is tilted upwards after decanting. During operation the channel and pour spout are filled with the decanted fluid. As the container is tilted upwards and the flow slows and eventually ceases, any final drip that may remain in the pour spout is captured by surface tension between the liquid and the walls of the channel, thereby inhibiting the last drip from falling off the edge of the pour spout.

The type of drip-less lip utilized can depend upon the viscosity of the liquid being decanted. A CHG composition of the subject invention can have a low viscosity, meaning that it can pour easily and leaves minimal surface residue. A bottle containing a CHG composition of the subject invention can employ a drip-free lip that can also minimize contact with the exterior of the container. In a particularly embodiment, the pour spout PS of a bottle of the subject invention can be a sharp edge 75, such as shown in FIG. 1D. The sharp edge can cause an immediate cessation in fluid flow, thereby decreasing incidence of drips. The sharp edge can also discourage the CHG composition from migrating towards the exterior of the bottle, thereby ensuring that the CHG composition decants substantially directly from the bottle interior to the splash basin.

In a further embodiment, the sharp edge can be radially flared, a shown, for example in FIG. 1D, to form a transition zone 76 in which the flow of the CHG composition can diminish as the bottle is tilted upwards, further minimizing dripping and/or contact with the exterior of the bottle.

As mentioned above, CHG can be provided in a concentrated form. In one embodiment, concentrated CHG can be provided in pre-measured bottles, such as, by way of example, an ampoule. The pre-measured, concentrated CHG can be added to a bottle or container of fluid, such as, for example, a bottle of sterile water, to form a diluted CHG composition prior to be decanted into a splash basin. Alternatively, the concentrated, pre-measured CHG can be added directly to a splash basin before, during, or after the splash basin is filled with a pre-determined amount of fluid, such as, for example, sterile water, to form a diluted CHG composition in the splash basin.

In one embodiment, the concentrated CHG is provided in pre-measured amounts. In a further embodiment, the pre-measured concentrated CHG is provided in a bottle 10 that can be opened to add CHG to a fluid, such as, for example, sterile water, and mixed prior to being decanted into a splash basin. In a particular embodiment, the pre-measured, concentrated CHG is a liquid provided an ampoule.

In a specific embodiment, the ampoule can contain approximately 30 ml to approximately 60 ml of concentrated CHG. In further specific embodiment, the concentrated CHG in an ampoule can be added to a one liter bottle of sterile water to obtain a diluted CHG composition having a final CHG concentration of about 1% w/v. In an alternative embodiment, the concentrated CHG in an ampoule is mixed with another fluid to provide a diluted CHG composition having a CHG concentration of less than 0.5% w/v, 0.1% w/v, 0.05% w/v, or 0.03% w/v, or a w/v in a range between any of the listed values.

It can be advantageous if the ampoule can be easily opened, particularly while wearing surgical gloves. In one embodiment, the ampoule has a non-replaceable, twist-off neck 17 that can be turned until it breaks or snaps the top or removable closure 12 off of the ampoule for form a mouth 21. For example, FIG. 5A illustrates an embodiment of an ampoule comprising a plastic or other deformable material, such that the neck can be turned or twisted until it separates from the bottom bottle or container portion. As shown in FIG. 5A, there can be flanges or wings 19 that assist in grasping the top of the ampoule for aiding in the twisting motion. Alternatively, an ampoule can have a removable closure 12 that is a with threading that can be unscrewed from the ampoule, one example of which is shown in FIG. 5B.

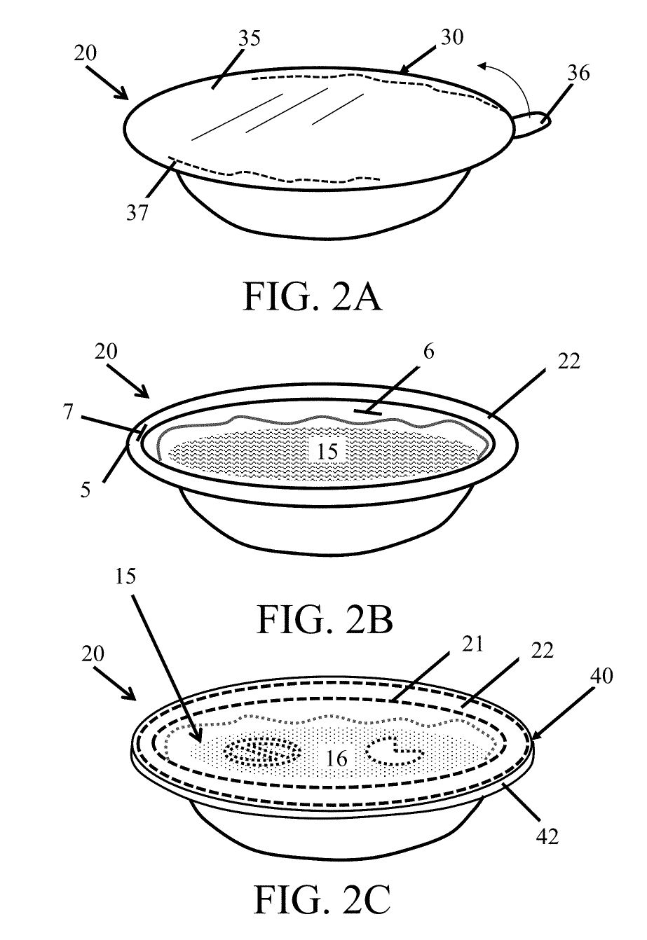

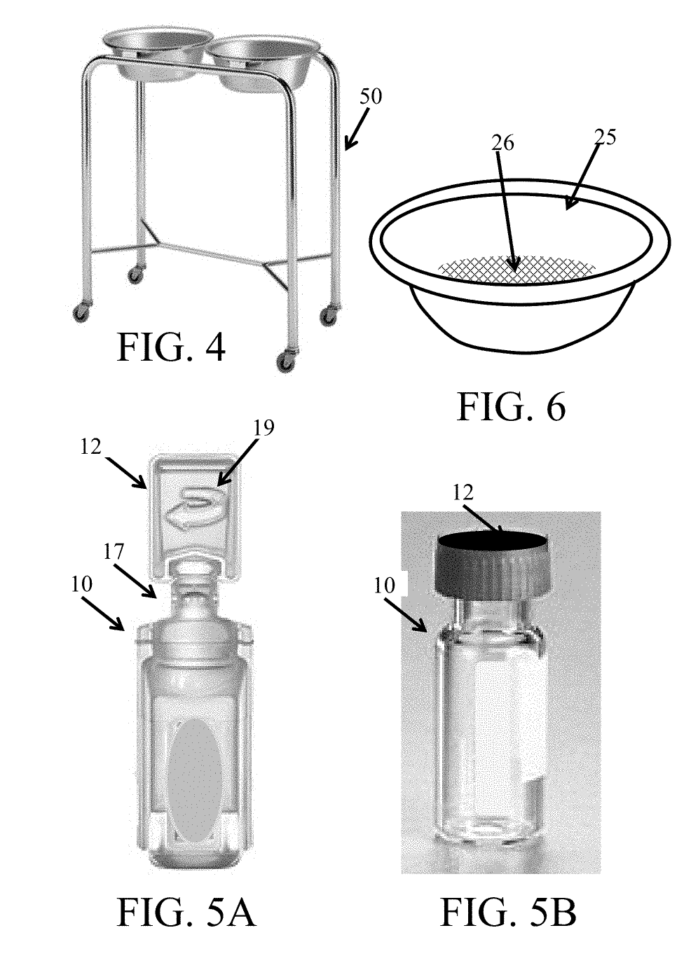

An alternative embodiment of the subject invention is a pre-filled splash basin (PFS basin) 20. In a further embodiment, the PFS basin is filled with a CHG composition of the subject invention. A PFS basin can be configured with a shape similar to or compatible with the splash basins currently used in operating rooms. For example, a PFS basin can have a lip 22 around the periphery of a mouth 21 that leads to a basin interior 6, such as shown in FIG. 2B. This can allow the PFS basin to be set into a ring stand 50, such as shown in FIG. 4, like other splash basins. Alternatively, the PFS basin can be set into another splash basin for support.

Another alternative embodiment of the subject invention is a splash basin that is coated with CHG to form a pre-coated splash basin (PCS basin). With a PCS basin, fluid can be decanted into the basin so that it dissolves or otherwise removes the CHG coating 26 so that it mixes with the fluid in the basin to create a diluted CHG composition. Preferably, a pre-determined amount of fluid is decanted so as to provide a diluted CHG composition of the desired concentration. For example, the coating can be applied such that decanting two liters of sterile water into the PCS basin will provide a diluted CHG composition of 1% w/v. If a lower w/v percentage is desired, additional sterile water can be added to the PCS basin to further dilute the CHG. In a further embodiment, the CHG coating 26 can be coated onto the bottom of the basin, so that as soon as a fluid is decanted it can start mixing with the CHG coating. FIG. 6 illustrates an example of a PCS basin with a coating of CHG on the bottom inside.

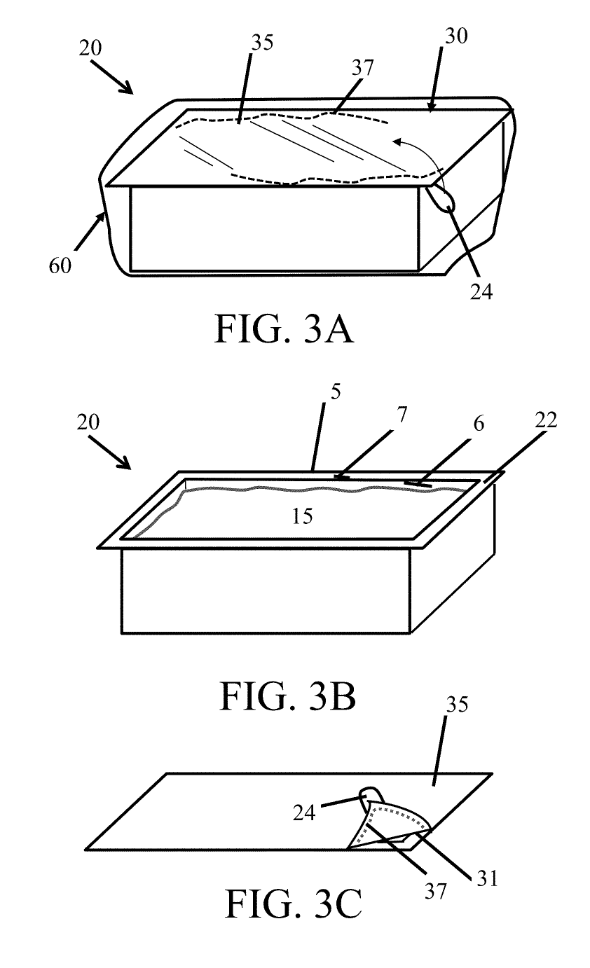

In another alternative, a PFS basin can have a rigid construction, such that it does not have to be supported and can stand alone on a surface. With this embodiment, a PFS basin could be any of a variety of shapes. FIGS. 3A and 3B illustrate a non-limiting example of a PFS basin that is not rounded, but is square or rectangular. Other polygonal shapes could also be used. This provides an opportunity to customize the splash basin for different procedures and different sizes and types of instruments.

A PFS basin can be manufactured from any of a variety of materials. It can be beneficial if the material is light-weight. It can be further advantageous if the material imparts rigidity or semi-rigidity to the shape, so that the PFS basin holds a shape and does not collapse, sag, bend, buckle, or otherwise deform either during storage or in use.

In one embodiment, the PFS basin is metal, such as, for example, stainless steel, aluminum, or another metal or metal alloy approved for hospital use. In an alternative embodiment, the PFS basin is a non-metallic material. There are several plastics, polymers, resins, and other non-metal materials that can be utilized for a PFS basin. Preferably, the one or more materials selected for a PFS basin, or components thereof, are capable of withstanding standard hospital sterilization procedures for containers. A person with skill in the art can determine the appropriate material for a PFS basin. Such variations are within the scope of this invention.

The PFS basin can also be sealed to maintain sterility. In one embodiment, the mouth 21 of the PFS basin is covered with a lid 30 that can be removed from the mouth. Any of a variety of removable lids, known to those with skill in the art, can be used. Preferably, the lid is capable of maintaining sterility of the interior 6 and any contents, can withstand routine hospital sterilization procedures for containers, and is easily removed from the mouth.

In one embodiment, the mouth 21 of the PFS basin is covered with a peel-away lid 35 that is attached to the upper surface 7 of the lip 22 of the PFS basin 20. FIGS. 2A and 3A illustrate non-limiting examples of a peel-away lid attached to a PFS basin. A tab 36 can be operably connected to the lid to facilitate grasping and pulling of the peel-away lid.

In a further embodiment, the peel-away lid can be attached to the upper surface 7 of the lip 22, so that the line of separation 31, where the lid separates from the lip, is generally parallel to the upper surface, as indicated in FIG. 3C, for a smoother release. Peel-away lids are known in the art and can be, for example, heat-sealed, pressure-sealed, glued, annealed, or otherwise attached by methods and techniques known in the art. In one embodiment, the peel-away lid can be single-use and cannot be reattached. In an alternative embodiment, the peel-away lid can have an adhering material 37 thereon that allows it to be at least partially reattached or stuck back onto the lip of the PFS basin. For example, a peel-away lid can have a glue, plastic, polymer or other sticky, adhesive, or self-attaching material that allows the lid to be at least put back in place over the mouth. This can be advantageous when transporting a PFS basin full of used composition 16, to inhibit splashing, dripping, or other loss of materials or debris from the basin.

In another embodiment, the mouth 21 of the PFS basin is covered or closed with a sealing lid 40 that operably attaches to the lip and/or sides of the PFS basin, so that the lid is both easily removable and replaceable over the mouth. The sealing lid can close the mouth and seal the peripheral edge and or the upper surface of the lip to inhibit spilling or splashing of contents. This can be particularly beneficial when sealing or closing a PFS basin full of used CHG composition.

In one embodiment, the lid has an overhang 42 that fits around the periphery 5 of the lip 22, such as illustrated, for example, in FIG. 2C. The overhang can be configured to engage with the lip by a friction fit with the lip. Alternatively, the overhang can engage with the lip by being conformable around the lip, such that one or more sections or areas of the overhang can be bent or folded under the lip to hold the sealing lid in place over the mouth. In yet another alternative, there can be a “seal and track” configuration where a seal on the lid fits into a track on the lip, or vice versa. These and other types of sealing lids are known in the art and a person with skill in the art could determine the most appropriate lid to be used with a PFS basin of the subject invention.

In a particular embodiment, a PFS basin utilizes both a peel-away lid and a sealing lid. With this embodiment, the sealing lid could be placed over a peel-away lid attached to the lip. The sealing lid can be removed and set aside for further use. The peel-away lid can be removed and discarded. The sealing lid can be replaced over the PFS basin to seal the contents and inhibit loss thereof, such as, as shown, for example, in FIG. 2C. For example, it can be beneficial to replace the sealing lid over the mouth of a used PFS basin.

In another embodiment, a PFS basin can be covered with a wrapping 60, such as, by way of non-limiting example, a peel-pack wrap where two sides of the container are peeled apart. A wrapping can be used alone or as an additional seal with one or more those described above. In one embodiment, the wrapping can be autoclaveable, allowing it to be brought into the sterile field. FIG. 3A illustrates a non-limiting example of a PFS basin with a peel-away lid that is further covered with a wrapping. A sealable lid could also be incorporated into this system.

With these embodiments of a PFS basin, it is possible to easily and quickly replace a PFS basin during a procedure or utilize more than one PFS basin in an operating room. By way, of example, a first PFS basin can be used until a pre-determined number of instruments have been deposited therein. In another example, a different PFS basin can be used for different types or differently used instruments. In yet another example, a PFS basin can be replaced after a pre-determined amount of time. In still another example, instruments can be placed in a first PFS basin for an initial rinse and can be removed and placed in a second PFS basin. When one or more of these methods are utilized with a CHG composition of the subject invention, opportunities for contamination can be reduced or inhibited.

All patents, patent applications, provisional applications, and other publications referred to or cited herein are incorporated by reference in their entirety, including all figures and tables, to the extent they are not inconsistent with the explicit teachings of this specification. Additionally, the entire contents of the references cited within the references cited herein are also entirely incorporated by reference.

The examples and embodiments described herein are for illustrative purposes only and that various modifications or changes in light thereof will be suggested to persons skilled in the art and are to be included within the spirit and purview of this application.

Any reference in this specification to “one embodiment,” “an embodiment,” “example embodiment,” “further embodiment,” “alternative embodiment,” etc., is for literary convenience. The appearance of such phrases in various places in the specification does not necessarily refer to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A device, adapted to inhibit contamination of a splash basin, the device comprising:
a non-gurgle container comprising;
an interior;
a mouth integral with the interior and having a drip-free lip;
a removable closure for sealing the mouth; and
a composition comprising chlorhexidine gluconate, at a w/v concentration of 1% or less, contained within the interior for decanting through the mouth upon removal of the closure.

2. The device according to claim 1, wherein the chlorhexidine gluconate composition has a concentration of chlorhexidine gluconate of between approximately 0.1% w/v and approximately 0.5% w/v.

3. A device, adapted to inhibit contamination of a splash basin, the device comprising:
a container comprising;
an interior;
a mouth integral with the interior;
a removable closure for sealing the mouth and having a twist-off neck; and
a composition comprising concentrated chlorhexidine gluconate contained within the interior for decanting through the mouth upon removal of the closure.

4. The device according to claim 3, wherein the container is an ampoule.

5. A method for inhibiting contamination of surgical instruments in a splash basin comprising:
A. obtaining a device, according to claim 1;
B. removing the removable closure;
C. decanting the chlorhexidine gluconate composition over the drip-free lip into a splash basin within an operating room; and
D. depositing one or more surgical instruments in the splash basin so as to be at least partially submerged in the chlorhexidine gluconate composition.

6. A method for inhibiting contamination of surgical instruments in a splash basin comprising:
A. obtaining a device, according to claim 3;
B. twisting the twist-off neck until the removable closure is removed;
C. decanting the chlorhexidine gluconate composition from the interior into a pre-determined amount of fluid to form a chlorohexidine gluconate composition for use in a splash basin;
D. depositing one or more surgical instruments into the splash basin containing the chlorhexidine composition, so as to be at least partially submerged.
